(19) United States
(12) Patent Application Publication
(10) Pub. No.: US 2007/0184474 A1
(43) Pub. Date: Aug. 9, 2007

(54) Process for amplifying DNA

(75) Inventors: Takeshi Koizumi, Tokyo (JP); Yoko Hamano, Chiba-shi (JP); Satoshi Yamamoto, Chiba-shi (JP)

Correspondence Address: COHEN, PONTANI, LIEBERMAN & PAVANE, 551 FIFTH AVENUE, SUITE 1210, NEW YORK, NY, 10176, US

(73) Assignee: Nichirei Corporation, Tokyo (JP)

(21) Appl. No.: 11/644,660

(22) Filed: Dec. 22, 2006

Related U.S. Application Data
(63) Continuation-in-part of application No. 10/601,713, filed on Jun. 20, 2003, now abandoned.

(30) Foreign Application Priority Data
Jan. 10, 2002 (JP) 2002-003912

(51) Int. Cl. C12Q 1/68; C12P 19/34; C07H 21/04

(52) U.S. Cl. 435/6; 435/91.2; 536/25.32

(57) ABSTRACT

The present invention has an object of providing a process for amplifying DNA. The present invention provides a process for amplifying DNA, comprising providing a primer in which a compound such as LC-Red 705 is added to the 5′ terminus; and amplifying said target DNA fragment via PCR using said PCR primer, in which annealing is carried out at the temperature higher than normal PCR reaction, and/or with the annealing time shorter than normal PCR reaction

### CROSS REFERENCE TO RELATED APPLICATIONS

This application is a Continuation-in-Part of U.S. patent application Ser. No. 10/601,713, filed Jun. 20, 2003, which claims priority from Applications filed in Japan on Jan. 10, 2002, No. 2002-003912 respectively.

### BACKGROUND OF THE INVENTION

1. Field of the Invention

The present invention relates to a process for amplifying DNA.

2. Description of Related Art

DNA amplification is extremely important in the detection of genes, and within the field of DNA amplification, the PCR method enables a large amplification of a targeted portion of nucleotide sequences within the DNA, and is a method which is used not only within biotechnology, but also within a variety of other fields.

However, when a specific detection primer is designed, the primer must include a base position specific to the target sequence.

Unfortunately, sequences including this type of position are frequently unsuitable as primers. In other words, in cases in which, for example, the AT content is extremely high, or the forward and reverse melting temperatures (Tm) do not match, the efficiency of the amplification deteriorates, making the sequence impractical for use as a primer. This problem becomes a considerable drawback in cases in which very small quantities of DNA need to be detected.

### BRIEF SUMMARY OF THE INVENTION

The inventors of the present invention discovered an extremely surprising fact. Namely, when a compound such as LC-Red 705, to the 5′ terminus of the degenerate primer, then the PCR amplification efficiency and specificity could be improved, resulting in an improvement in the efficiency and specificity of the DNA amplification reaction, and the inventors were hence able to complete the present invention. The optimum temperature range for annealing could be widened, meaning the preliminary tests for investigating the annealing conditions could be simplified, the overall process could be simplified considerably.

In other words, the present invention provides a process for amplifying a target DNA fragment comprising:

providing a PCR primer that comprises a compound at the 5′ terminus, said compound selected from a group consisting of LC-Red 705, an amino group, a phosphate group, DIG, DNP, TAMRA, Texas-Red, ROX, XRITC, rhodamine, LC-Red 640, a mercapto group, psoralen, cholesterol, FITC, 6-FAM, TET, cy3, cy5, BODIPY 564/570, BODIPY 500/510, BODIPY 530/550, BODIPY 581/591 (hereafter described as the “specified compounds group”); and

amplifying said target DNA fragment via PCR using said PCR primer, in which annealing of the primer to the target DNA is carried out at the temperature higher than normal PCR reaction, and/or with the annealing time shorter than normal PCR reaction.

In the present invention, the annealing of the primer to the target DNA can be carried out at the temperature higher than normal PCR reaction, more specifically, at the temperature at which a PCR product in a detectable amount is obtained for a primer with a specified compound but a PCR product in a significantly smaller amount is obtained for a primer without said compound. In the present invention, the annealing of the primer to the target DNA can be also carried out for an annealing time shorter than normal PCR reaction, more specifically, the time with which a PCR product in a detectable amount is obtained for a primer with a specified compound but a PCR product in a significantly smaller amount is obtained for a primer without said compound.

In the present invention, the annealing temperature higher than normal PCR reaction is at least 1.7° C. higher than the Tm of a primer without said compound. The Tm can be calculated according to one of the Nearest Neighbor Method, the Wallace Method, or the GC % Method.

### BRIEF DESCRIPTION OF THE DRAWINGS

FIG. 1 is a graph showing the relationship between the number of PCR cycles and the fluorescence intensity in annealing conditions of 0 seconds at 60° C.

FIG. 2 is a graph showing the relationship between the number of PCR cycles and the fluorescence intensity in annealing conditions of 5 seconds at 60° C.

FIG. 3 is a graph showing the relationship between the number of PCR cycles and the fluorescence intensity in annealing conditions of 10 seconds at 60° C.

FIG. 4 is a graph showing the relationship between the number of PCR cycles and the fluorescence intensity in annealing conditions of 5 seconds at 64° C.

### DETAILED DESCRIPTION OF THE INVENTION

In the present invention, there are no particular restrictions on the primer to which the compound selected from the specified compounds group is conjugated or added, nor on the oligonucleotide to which the compound selected from the specified compounds group is conjugated, provided they represent a primer or an oligonucleotide which is typically used in DNA amplification. Furthermore, because the present invention enables the DNA amplification efficiency to be improved, some primers which are not normally usable for DNA amplification can also be used.

In the present invention, the compounds of the specified compounds group are non-specific with respect to the target sequence to be amplified by DNA amplification. DIG is an abbreviation for digoxigenin, DNP is an abbreviation for dinitrophenyl, TAMRA refers to carboxytetramethylrhodamine, Texas-Red is 1H,5H,11H,15H-Xantheno[2,3,4-ij:5,6,7-i′j′]diquinolizin-18-ium, 9-[2 (or 4)-[[[6-(2,5-dioxo-1-pyrrolidinyl)oxy]-6-oxohexyl]amino]sulfonyl]-4 (or 2) sulfophenyl]-2,3,6,7,12,13,16,17-octahydro-, inner salt, ROX is an abbreviation for rhodamine X, XRITC refers to rhodamine X isothiocyanate, FITC is an abbreviation for fluorescein isothiocyanate, 6-FAM refers to 6-carboxyfluorescein, TET is an abbreviation for tetrachlorofluorescein, BODIPY 564/570 is 4,4-difluoro-5-styryl-4-bora-3a,4a-diaza-s-indacene-3-propionic acid, succinimidyl ester, BODIPY 530/550 is 4,4-difluoro-5,7-diphenyl-4-bora-3a,4a-diaza-s-indacene-3-propionic acid, succinimidyl ester, and BODIPY 581/591 is 4,4-difluoro-5-(4-phenyl-1,3-butadienyl)-4-bora-3a,4a-diaza-s-indacene-3-propionic acid, succinimidyl ester.

In the present invention, either one, or two or more compounds selected from the specified compounds group can be used.

In the present invention, the primer and the compound selected from the specified compounds group may also be conjugated via a linker. Examples of suitable linkers include hydrocarbon groups of 2 to 16 carbon atoms.

In the present invention, the compound selected from the aforementioned specified compounds group can be conjugated or added to the 5′ terminus of the primer in accordance with standard methods.

In the present invention, by conjugating a compound selected from the specified compounds group to a primer, both the annealing speed of the primer to the amplified product and the annealing stability can be improved, meaning the primer is ideally suited to normal PCR. In addition, the present invention can also be ideally applied to asymmetric PCR.

Asymmetric PCR is a method used for rapidly amplifying a single strand DNA, such as in cases where a target DNA fragment needs to be directly sequenced. In other words, whereas in normal PCR the concentrations of the pair of primers used are equal, in asymmetric PCR, the concentration of one of the primers is raised to several times, or several dozen times that of the other primer. By so doing, the lower concentration primer is consumed first, and the remaining PCR proceeds only from the residual higher concentration primer, producing a large quantity of the DNA strand corresponding with the higher concentration primer. Furthermore, in thermal asymmetric PCR, which represents one specific type of asymmetric PCR, a pair of primers is used which display a difference in Tm of at least 10° C., and first PCR is conducted under conditions in which the primer with the lower Tm value will also undergo annealing, and subsequently PCR is conducted under conditions in which only the primer with the higher Tm value will undergo annealing.

However, asymmetric PCR suffers from the types of problems described below. Namely, if the concentrations of the template DNA and the primers are not optimized, then the amplification of the single strand is low (Production of Single Stranded DNA by Asymmetric PCR, PCR Protocols, A guide to Methods and Applications, Academic Press, Inc. 1990). However, such optimization requires complex preliminary tests.

Furthermore in thermal asymmetric PCR, a set of specific primers with a large difference in annealing temperature of at least 10° C. must be prepared, and this is not necessarily a simple task.

Another method of rapidly amplifying a single strand DNA utilizes the difference in amplification ability within a pair of primers. For example, hybrid primers of DNA and RNA can be used. RNA primers display a weaker contribution to extension reactions than DNA primers, and consequently if PCR is conducted with these types of hybrid primers, then the amplification at the pure DNA side will be larger, yielding a single strand DNA.

However, this method results in a single strand DNA due to the low amplification ability of the RNA side, and does not result from any improvement in the amplification ability of the desired DNA.

In contrast, in the present invention, by conjugating a compound selected from the specified compounds group to only one of the pair of primers, a large difference in amplification efficiency can be generated between the two primers, meaning the complex operations of optimizing the concentrations of the template DNA and the primers are not required. Consequently, by applying the present invention to asymmetric PCR, the amplification efficiency for a single strand DNA can be improved markedly. Furthermore, the present invention also enables the optimum temperature range for the primers to be widened, making the invention also applicable to thermal asymmetric PCR.

Conventionally, asymmetric PCR has been conducted by inhibiting the extension of one of the primers, that is, by effectively lowering the overall PCR efficiency. In contrast, the present invention enables asymmetric PCR to be conducted by improving the amplification efficiency of one of the primers. In other words, when compared with conventional PCR, asymmetric PCR using the present invention suffers no reduction in amplification efficiency. Accordingly, the present invention is particularly effective in those cases in which generation of a single strand DNA is required while maintaining a high level of amplification efficiency, such as the case in which a minute quantity of DNA such as a pathogen needs to be detected and typed rapidly and easily.

In addition, the present invention can also be ideally applied to degenerate PCR. In degenerate PCR, a mixture of between several hundred and several thousand different primers are used, meaning that the optimum annealing temperature will differ for each of the sequences within the mixture. As a result, the setting of the amplification temperature is comparatively difficult. However, this type of problem can also be resolved using the present invention. Moreover, because the annealing temperature can be set to a relatively high temperature, non-specific amplification can be suppressed, enabling a more efficient amplification.

A primer to which a compound selected from the specified compounds group of the present invention has been conjugated can also be used as a probe.

In the present invention, annealing reaction of a primer to target DNA can be carried out at the temperature higher than normal PCR, and/or with the annealing time shorter than normal PCR reaction. The temperature for carrying out annealing reaction in the present invention is such that a PCR product in a detectable amount is obtained for the primer with the specified compounds of the present invention, but a PCR product in a significantly smaller amount is obtained for a primer without the specified compounds of the present invention. More specifically, the annealing temperature in the present invention is at least 1.7° C., preferably 2.0° C., more preferably 3.0° C. higher than the Tm of a primer without the specified compound attached to its 5′ terminus. The Tm of a primer without the specified compound can be calculated according to any one of the following methods: the Nearest Neighbor Method, the Wallace Method, and GC % Method.

The Nearest Neighbor Method:
Tm(° C.)=1000ΔH/(−10.8+ΔS+Rln(Ct/4))−273.15+Log [Na⁺]
ΔH: the sum of a change in the nearest enthalpy in the hybrid
ΔS: the sum of a change in the nearest entropy in the hybrid
−10.8: ΔS (initiation) [cal/mol·k]
R: Gas constant
Ct: Molar of primers [mol/l]
[Na⁺]: The molar of Na⁺[mol/l]
   The Wallace Method
   Tm(° C.)=4(G+C)+2(A+T)
G+C: the numbers of nucleotides, G and C in the primer
A+T: the numbers of nucleotides, A and T in the primer
   The GC % Method
   Tm(° C.)=81.5+16.6*log [S]+0.41*(% GC)−(500/n)
[S]: Molar of a salt
(% GC): GC content in the primer (%)
n: Length of the primer (bp)

Among these three methods, the Nearest Neighbor Method is preferred in terms of accuracy, and the Wallace Method is preferred in terms of simplicity.

In the normal PCR, the annealing temperature of a primer to a target DNA is usually set to the Tm, which is calculated based on the sequence information of the primer and one of the three methods described above. If a PCR product cannot be obtained with the Tm, the annealing temperature is lowered; and if a nonspecific annealing occurs at the Tm, the annealing temperature is raised. In either case, lowering or raising the temperature is usually conducted by 2° C. at a time, that is the annealing temperature afoter adjustment is Tm−2*n or Tm+2*n (n=1, 2, 3 . . . ).

The annealing time shorter than normal PCR in the present invention is such that a PCR product in a detectable amount is obtained for the primer with the specified compounds of the present invention, but a PCR product in a significantly smaller amount is obtained for a primer without the specified compounds of the present invention. Typically, the annealing time is set to be 30 sec, so the annealing time in the present invention can be 10 sec, preferably 5 sec, more preferably 0 sec.

### EXAMPLES

As follows is a more detailed description of the present invention based on a series of examples. However, the present invention is in no way restricted to the examples presented below.

### Example 1

Comparison of the Upper Limit Annealing Temperature for Amplification

Using primers for the detection of Vibrio parahaemolyticus with a compound selected from the specified compounds group conjugated to the 5′ terminus, and utilizing a PCR Express device manufactured by Hybaid Co., Ltd with a Gradient Block Module added, the upper limit annealing temperature for amplification was measured by conducting PCR under the conditions described below.

The primers for the detection of Vibrio parahaemolyticus utilized a nucleotide sequence represented by the sequence number 1 as the forward side primer and a nucleotide sequence represented by the sequence number 2 as the reverse side primer.
Sequence number 1: aagaagacct agaagatgat
Sequence number 2: gttaccagta atagggca
   Each of the compounds of the specified compounds group shown in Table 1 was conjugated to the 5′ termini of the forward side primer and the reverse side primer. In a separate preparation, chromosome DNA extracted from a type strain (IF012711T) of Vibrio parahaemolyticus was used as a template, and PCR was conducted using a rpoD gene amplification universal primer (refer to Japanese Unexamined Patent Application, Publication No. Hei 8-256798: sequence numbers 3 and 4) to prepare an amplified product. Subsequently, using this amplified product as a template, PCR tests were conducted under a plurality of different annealing temperature conditions, using the aforementioned primers with added compounds from the specified compounds group.
Sequence number 3: yatgmgngar atgggnacng t
   (y stands for a base T or U, or C; m stands for A or C; and n stands for A, C, G, or T or U)
Sequence number 4: ngcytcnacc atytcyttyt t

The PCR conditions were as follows. (1) Activation of Taq polymerase (AmpliTaq Gold, manufactured by Applied Biosystems Co., Ltd.): 10 minutes at 95° C. (2) Denaturation: 1 minute at 94° C. (3) Annealing: 30 seconds at 55.1° C., 55.5° C., 57.7° C., 59.4° C., 61.4° C., 63.3° C., 65.3° C., 67.6° C., 69.0° C., 69.7° C. and 70.2° C. (4) Extension reaction: 1 minute at 72° C. The above steps (2) through (4) were repeated for 40 cycles. (5) Extension reaction: 10 minutes at 72° C. (6) Cooling: cooled to 4° C.

Subsequently, the thus obtained amplified fragments were analyzed by agarose gel electrophoresis, and the upper limit annealing temperatures were determined. A primer with no conjugated compound from the specified compounds group was used as a control. The results for the upper limit annealing temperatures, and the temperature increases in the upper limit annealing temperatures relative to the control value, are shown in Table 1.

TABLE 1

| | | | | Temperature |
|---|---|---|---|---|
| | | | Upper | increase |
| | | | limit | (compar- |
| | | | annealing | ison with |
| Conjugated | | | temper- | control |
| Compound | Linker | Effect | ature | primer) |
| BODIPY564/570 | | AA (Max) | 63.3° C. | 5.6° C. |
| LC-Red 705 | none | AA | 63.3° C. | 5.6° C. |
| Amino group C3 | C3 | A | 61.4° C. | 3.7° C. |
| Phosphate group | none | A | 61.4° C. | 3.7° C. |
| Biotin | C10 | A | 61.4° C. | 3.7° C. |
| DIG | C6 | A | 61.4° C. | 3.7° C. |
| DNP | C14 | A | 61.4° C. | 3.7° C. |
| TAMRA | C6 | A | 61.4° C. | 3.7° C. |
| Texas-Red | C6 | A | 61.4° C. | 3.7° C. |
| ROX | C6 | A | 61.4° C. | 3.7° C. |
| XRITC (Rhodamine | C6 | A | 61.4° C. | 3.7° C. |
| 600) | | | | |
| Rhodamine | C6 | A | 61.4° C. | 3.7° C. |
| LC-Red 640 | C6 | A | 61.4° C. | 3.7° C. |
| BODIPY500/510 | | A | 61.4° C. | 3.7° C. |
| BODIPY530/550 | | A | 61.4° C. | 3.7° C. |
| BODIPY581/591 | | A | 61.4° C. | 3.7° C. |
| Amino group C6 | C6 | B | 59.4° C. | 1.7° C. |
| SH (thiol) | none | B | 59.4° C. | 1.7° C. |
| Psoralen C2 | C2 | B | 59.4° C. | 1.7° C. |
| Psoralen C6 | C6 | B | 59.4° C. | 1.7° C. |
| Cholesterol | | B | 59.4° C. | 1.7° C. |
| FITC | C6 | B | 59.4° C. | 1.7° C. |
| 6-FAM | C6 | B | 59.4° C. | 1.7° C. |
| TET | C6 | B | 59.4° C. | 1.7° C. |
| cy3 | C3 | B | 59.4° C. | 1.7° C. |
| cy5 | C3 | B | 59.4° C. | 1.7° C. |
| No label (Control) | | — | 57.7° C. | — |

AA: Excellent
A: Good
B: Fair

The primers with added compounds from the specified compounds group displayed an increase in the upper limit annealing temperature, and produced an improvement in amplification efficiency.

### Example 2

Investigation of the Amplification Efficiency in Primers Containing a Compound Selected from the Specified Compounds Group Added to the 5′ Terminus

A nucleotide sequence represented by the sequence number 1 was used as the forward side primer and a nucleotide sequence represented by the sequence number 2 was used as the reverse side primer. Either cy3, cy5, or biotin, were added (conjugated) to the 5′ termini of the forward side primer and the reverse side primer, and the kinetics of the amplification reaction were analyzed by conducting real time PCR under conditions described below, using a Light Cycler System (manufactured by Roche Diagnostics Co., Ltd.), and using chromosome DNA extracted from a type strain (IFO12711T) of Vibrio parahaemolyticus as a template.

The PCR conditions were as follows. (1) Denaturation: 1.5 minutes at 95° C. (2) Denaturation: 0 seconds at 95° C. (3) Annealing: 0, 5 or 10 seconds at 60° C., or 5 seconds at 64° C. (4) Extension reaction: 15 seconds at 72° C.

The above steps (2) through (4) were repeated for 40 cycles.

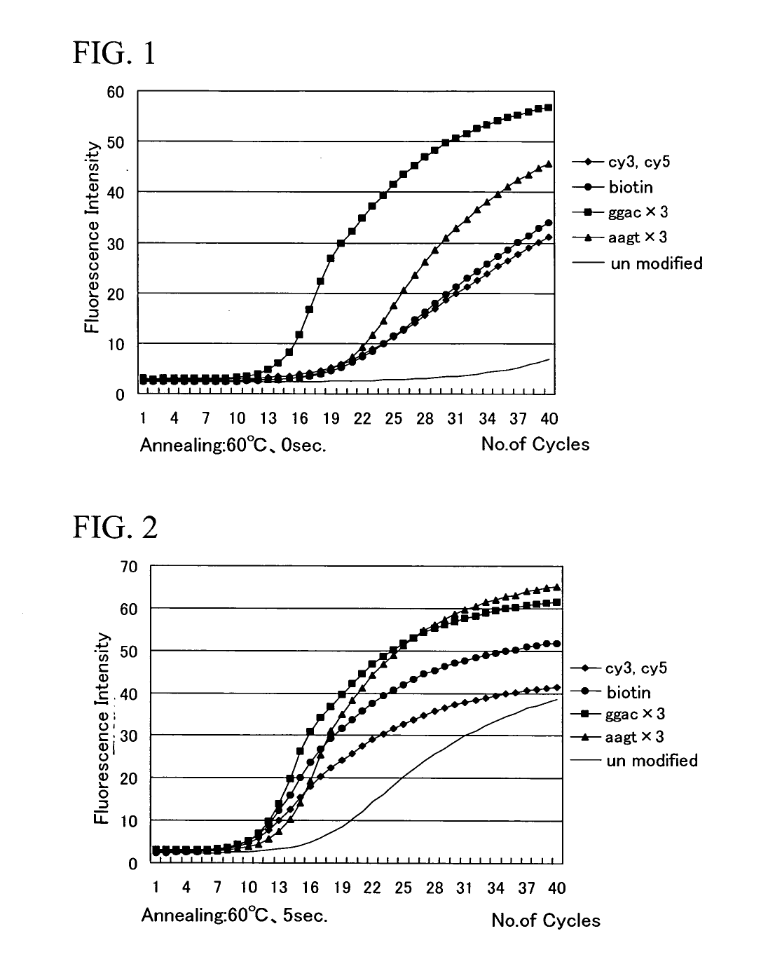

The amplified fragment obtained after each cycle was measured for fluorescence intensity. A primer with no added compound from the specified bases was used as a control. The results are shown in FIG. 1 through FIG. 4. This fluorescence intensity is not derived from the compounds of the specified compounds group conjugated to the primer, but rather is derived from cyber green intercalated to the double strands, and indicates the accumulated quantity of amplified DNA.

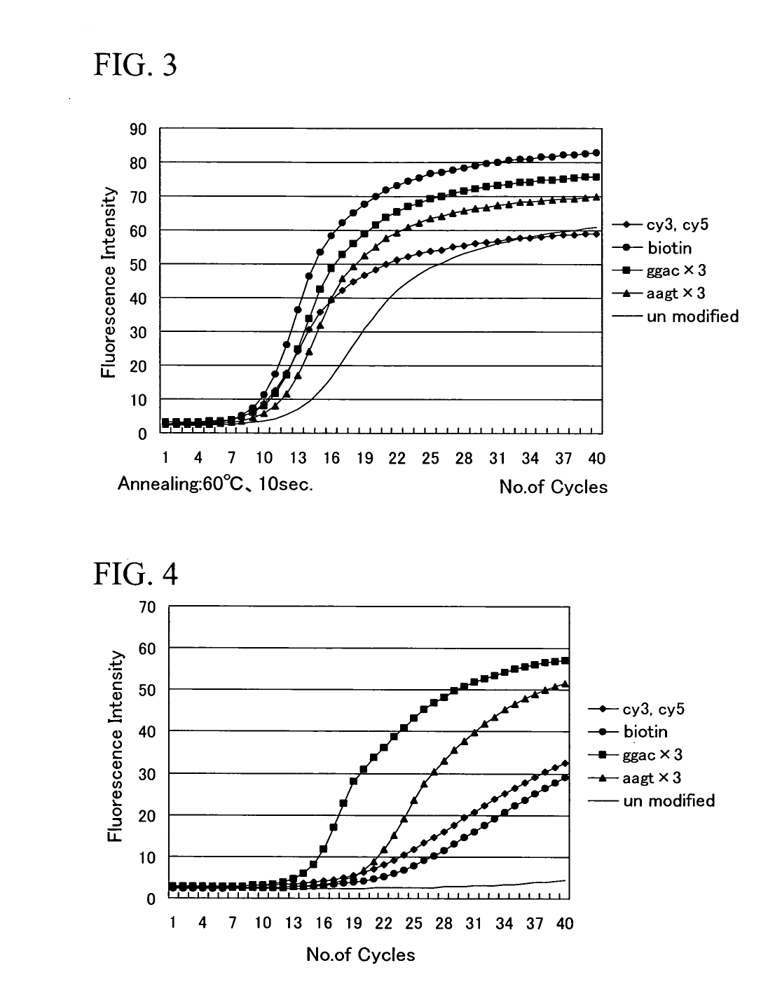

FIG. 1 is a graph showing the relationship between the number of PCR cycles and the fluorescence intensity in annealing conditions of 0 seconds at 60° C. FIG. 2 is a graph showing the relationship between the number of PCR cycles and the fluorescence intensity in annealing conditions of 5 seconds at 60° C. FIG. 3 is a graph showing the relationship between the number of PCR cycles and the fluorescence intensity in annealing conditions of 10 seconds at 60° C. FIG. 4 is a graph showing the relationship between the number of PCR cycles and the fluorescence intensity in annealing conditions of 5 seconds at 64° C.

Under all the amplification conditions, the primers with an added compound selected from the specified compounds group (the modified primers) displayed an earlier rise in the amplification reaction than the primer without an added compound selected from the specified compounds group or the specified bases (the unmodified primer). In other words, when the modified primers are used, the number of cycles required to achieve a constant fluorescence intensity shortens (refer to FIG. 2 and FIG. 3).

If the annealing time is shortened for the same annealing temperature (from 10 seconds (FIG. 3) to 5 seconds (FIG. 2) to 0 seconds (FIG. 1)), then the cycle at which amplification of the unmodified primer commences is delayed considerably (in other words, the amplification weakens). For example, in order to achieve a fluorescence intensity exceeding 10, 14 cycles are required in the case of an annealing time of 10 seconds (FIG. 3), whereas 20 cycles are required in the case of an annealing time of 5 seconds (FIG. 3), and in the case of an annealing time of 0 seconds, the fluorescence intensity does not exceed 10, even after 40 cycles. In contrast, in the case of the modified primers, the delay in the amplification commencement cycle resulting from shortening of the annealing time is considerably less than that observed for the unmodified primer. In other words, when used in a detection system, the modified primers provide an increase in sensitivity. This modification effect is particularly marked for the case of an annealing time of 0 seconds.

With the modified primers, a practical level of amplification can be achieved even using annealing temperatures and annealing times for which amplification does not occur with the unmodified primer (refer to FIG. 4).

The final quantity of the amplified product after 40 cycles is markedly higher for the modified primers than for the unmodified primer (refer to FIG. 1 through FIG. 4). In normal PCR, because the amplification reaction is not usually conducted beyond 40 cycles, the modified primers offer a distinct advantage within a practical cycle range.

The above results reveal a modification effect under conditions of both increased temperature and shortened annealing time, and it is thought that these effects are due to an improvement in the thermal stability of the hybridization between the primer and the template DNA, that is, an increase in the bonding strength.

According to the present invention, preliminary tests for investigating the annealing conditions and the like can be simplified considerably, and the PCR amplification efficiency can be improved. These effects are particularly marked in those cases in which the PCR is either asymmetric PCR or degenerate PCR.

What is claimed is:

1. A method for amplifying a target DNA fragment comprising:
providing a PCR primer that comprises a compound at the 5′ terminus, said compound selected from a group consisting of LC-Red 705, an amino group, a phosphate group, DIG, DNP, TAMRA, Texas-Red, ROX, XRITC, rhodamine, LC-Red 640, a mercapto group, psoralen, cholesterol, FITC, 6-FAM, TET, cy3, cy5, BODIPY 564/570, BODIPY 500/510, BODIPY 530/550, BODIPY 581/591; and
amplifying said target DNA fragment via PCR using said PCR primer, in which annealing of said primer to said DNA fragment is carried out at the temperature higher than normal PCR reaction, and/or with the annealing time shorter than normal PCR reaction.

2. A method for amplifying a target DNA fragment according to claim 1, wherein said annealing temperature higher than normal PCR reaction is the temperature at which a PCR product in a detectable amount is obtained for said primer but a PCR product in a significantly smaller amount is obtained for a primer without said compound; and
said annealing time is the time with which a PCR product in a detectable amount is obtained for said primer but a PCR product in a significantly smaller amount is obtained for a primer without said compound.

3. A method for amplifying a target DNA fragment according to claim 1, wherein said annealing temperature higher than normal PCR reaction is at least 1.7° C. higher than the Tm of a primer without said compound.

4. A method for amplifying a target DNA fragment according to claim 3, wherein said Tm is calculated according to one of the Nearest Neighbor Method, the Wallace Method, or the GC % Method.

5. A method for amplifying a target DNA fragment according to claim 1, wherein said PCR is either one of asymmetric and degenerate PCR.
